(19) United States
(12) Patent Application Publication
(10) Pub. No.: US 2017/0027707 A1
(43) Pub. Date: Feb. 2, 2017

(54) FEMORAL COMPONENT FOR KNEE PROSTHESES

(71) Applicant: ADLER ORTHO S.R.L., Cormano (IT)

(72) Inventor: Patrizio CREMASCOLI, Bresso (IT)

(21) Appl. No.: 15/106,743

(22) PCT Filed: Nov. 20, 2014
(86) PCT No.: PCT/EP2014/075122

§ 371 Date: Jun. 20, 2016

(30) Foreign Application Priority Data
Dec. 20, 2013 (IT) MI2013A002154

(51) Int. Cl. A61F 2/38; A61F 2/30

(52) U.S. Cl. CPC A61F 2/3859; A61F 2/3094; A61F 2002/30011; A61F 2002/3092; A61F 2002/3097

(57) ABSTRACT

A femoral component for knee prostheses includes a body of the femoral component that defines a supporting structure of the femoral component with mediolateral reinforcements and longitudinal reinforcements. A highly porous structure is arranged inside the body of the femoral component, and a porous structure is arranged as an interface between the femoral component and a bone. The femoral component is provided by EBM beam or laser beam powder technology.

### TECHNICAL FIELD

The present disclosure relates to a femoral component for knee prostheses. More particularly, the disclosure relates to a femoral component for the knee articulation with a porosity of a different nature and therefore of reduced weight.

### BACKGROUND

As is known, the artificial prosthesis for the knee joint is being developed continuously both in terms of the number of implantations carried out and in terms of research into increasingly high-performance prostheses.

The knee prostheses have reached such numbers as to equal and sometimes exceed the number of implanted hip prostheses.

The knee is the most important and complex joint of the lower limb. Although it has a broad range of movement, it has good stability by thanks to the presence of the ligamentous apparatuses. In the knee it is possible to recognize a bone component and a capsular apparatus with tendinous formations.

The needs for intervention on the knee with the implantation of a prosthesis are due mainly to arthrosis, rheumatoid arthritis, bone necrosis and neoplasms.

The knee prosthesis can be of the tricompartmental type, constituted in this case by the following components:

a) femoral component made of metal, which can be fixed to the bone both by press-fit (by pressure interlocking) and with acrylic cement. In the first case, the metallic surface in contact with the bone has a microfinish that allows good osteointegration of the bone wall with the prosthesis itself,

the second mode is cementing, i.e., the surgeon uses a sort of adhesive, known as acrylic cement (methyl methacrylate), and fixes the prosthesis to the receiving bone;

b) tibial component made of metal which is fixed to the bone, for example with acrylic cement (and optional screws);

c) inserts made of polyethylene (with extremely high molecular density, therefore very strong, interlocked on the upper surface of the tibial component. The insert acts as a shock absorber between two metallic components and facilitates their movement without metal wear.

d) optional rotular component fixed preferably by means of cement.

Known prostheses can also be of the unicompartmental or bicompartmental type.

One of the drawbacks of the femoral prosthesis is due to the weight of the femoral component of the prosthesis, which is necessary to ensure sufficient mechanical strength of the prosthesis itself.

### SUMMARY

The aim of the present disclosure is to provide a femoral component for knee prostheses that is of the cementless type and of the type with cement, unicompartmental, bicompartmental and tricompartmental, and has a porosity of a different nature and therefore a reduced weight with respect to femoral components of the known type.

Within this aim, the present disclosure provides a femoral component which, despite porosity of a different nature and therefore of a reduced weight with respect to femoral components of the known type, has a mechanical strength comparable to the one of the latter.

The present disclosure also provides a femoral component that is highly reliable, relatively simple to provide and has competitive costs.

These aims and others that will become more apparent hereinafter are achieved by providing a femoral component for cementless knee prostheses, characterized in that it comprises:

a body of the femoral component that defines the supporting structure of the femoral component with mediolateral or longitudinal reinforcements,

a highly porous structure arranged inside the body of the femoral component,

and a porous structure arranged as an interface between the femoral component and the bone,

said femoral component being provided by means of EBM beam or laser beam powder technology.

This aim is also achieved by a femoral component for knee prostheses, characterized in that it comprises:

a body of the femoral component that is adapted to define the supporting structure of the femoral component with mediolateral or longitudinal reinforcements,

a highly porous structure arranged inside said body, and

a substantially smooth structure arranged over the porous component and adapted to define the interface with the bone,

said femoral component being provided by means of EBM beam or laser beam powder technology.

### BRIEF DESCRIPTION OF THE DRAWINGS

Further characteristics and advantages of the disclosure will become more apparent from the description of preferred but not exclusive embodiments of the femoral component according to the present disclosure, illustrated by way of non-limiting example in the accompanying drawings, wherein:

FIG. 1 is a perspective view of a first embodiment of the femoral component according to the present disclosure, for cementless application;

FIG. 2 is a perspective view, from a different angle than in FIG. 1, of the femoral component according to the present disclosure;

FIG. 3 is a partially cutout sectional front view of the femoral component of FIGS. 1 and 2;

FIG. 4 is a partially sectional view of the femoral component according to the present disclosure;

FIG. 5 is an exploded perspective view of the femoral component according to the present disclosure;

FIG. 6 is a perspective view of a second embodiment of a femoral component according to the present disclosure, for application with cement; and

FIG. 7 is an embodiment of the femoral component according to the disclosure, with longitudinal reinforcements.

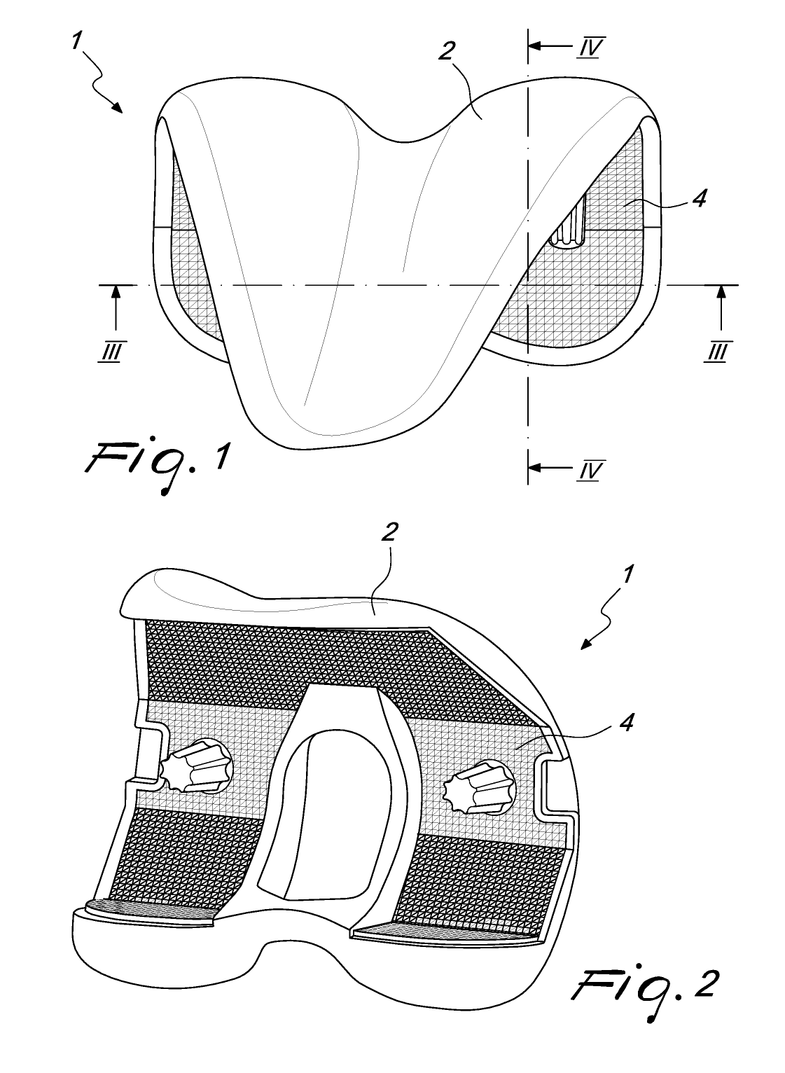

### DETAILED DESCRIPTION OF THE DRAWINGS

With reference to the figures, the femoral component for knee prostheses according to the disclosure, generally designated by the reference numeral 1, comprises, in the first embodiment, in which the femoral component is fixed without cement, a body 2 of the femoral component that defines the supporting structure of the femoral component, which can be of the unicompartmental, bicompartmental and tricompartmental type, and is provided by means of powder technology, for example with an EBM (Electron Beam Melting) beam or laser beam.

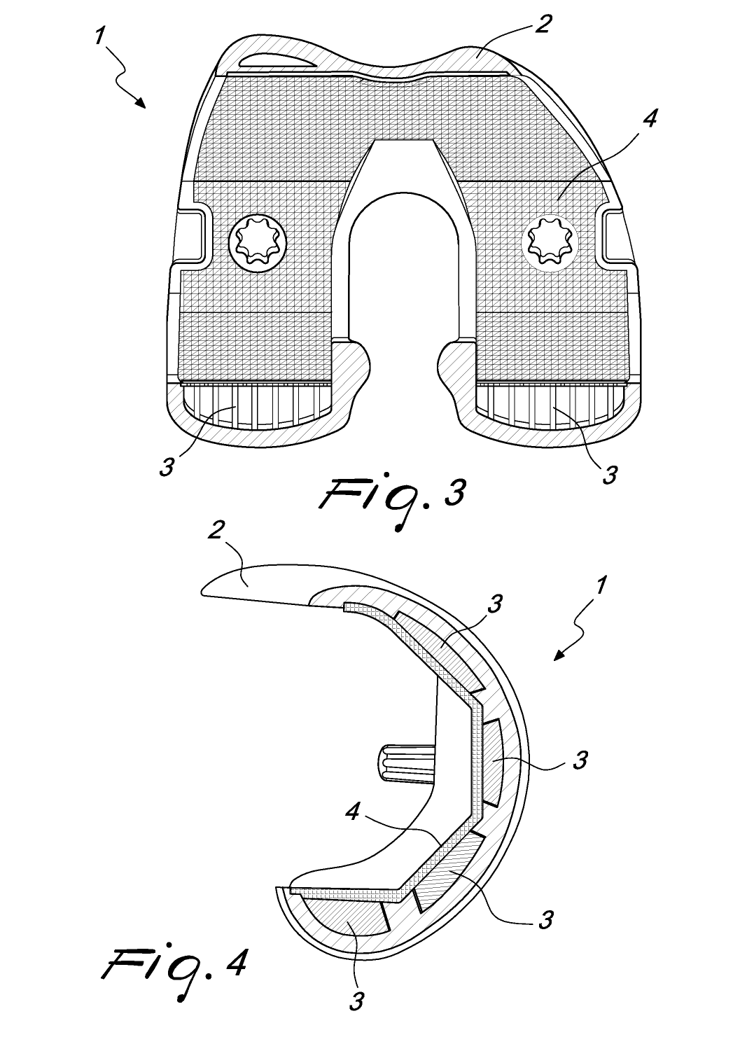

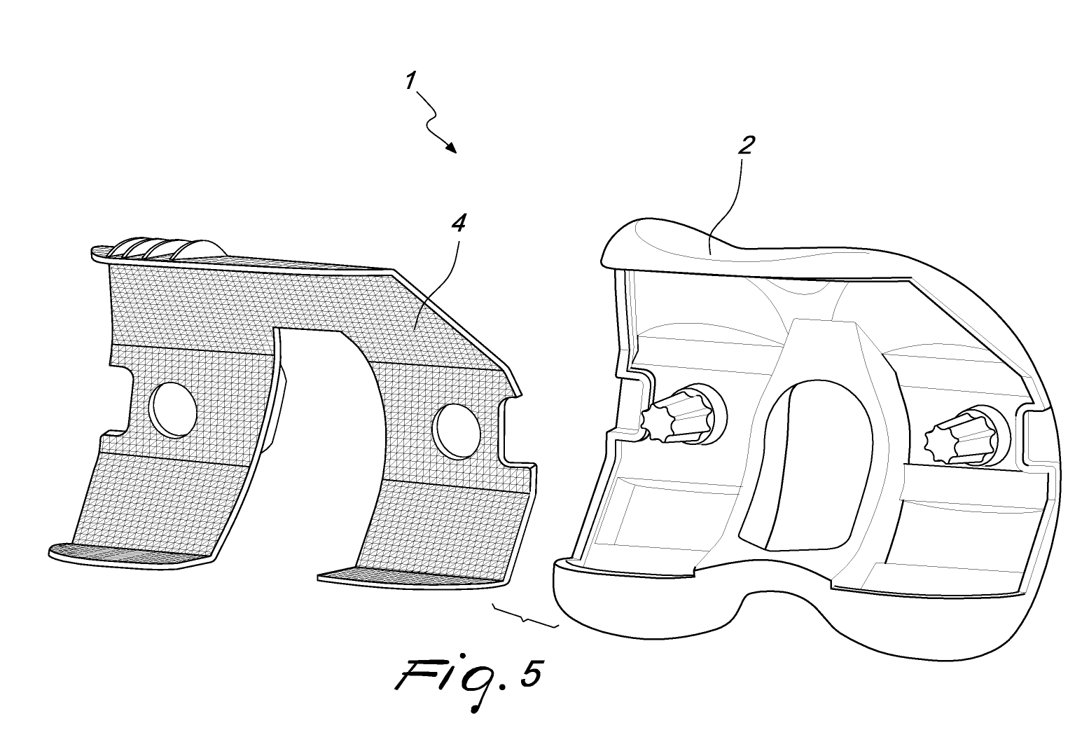

The body 2 is provided with mediolateral reinforcements (see FIG. 4) or longitudinal reinforcements (as shown in FIG. 7).

The femoral component is conveniently made of cobalt chromium or other suitable material.

The internal part of the body 2 thus provided is coated with a highly porous structure (with a porosity of more than 1000 microns) on which a porous structure 4 with a porosity comprised between 300 and 1,000 microns is arranged at the interface between the femoral component and the bone.

Both the highly porous structure 3 and the porous structure 4 are provided, like the body 2 of the prosthesis, by means of powder technology as explained above.

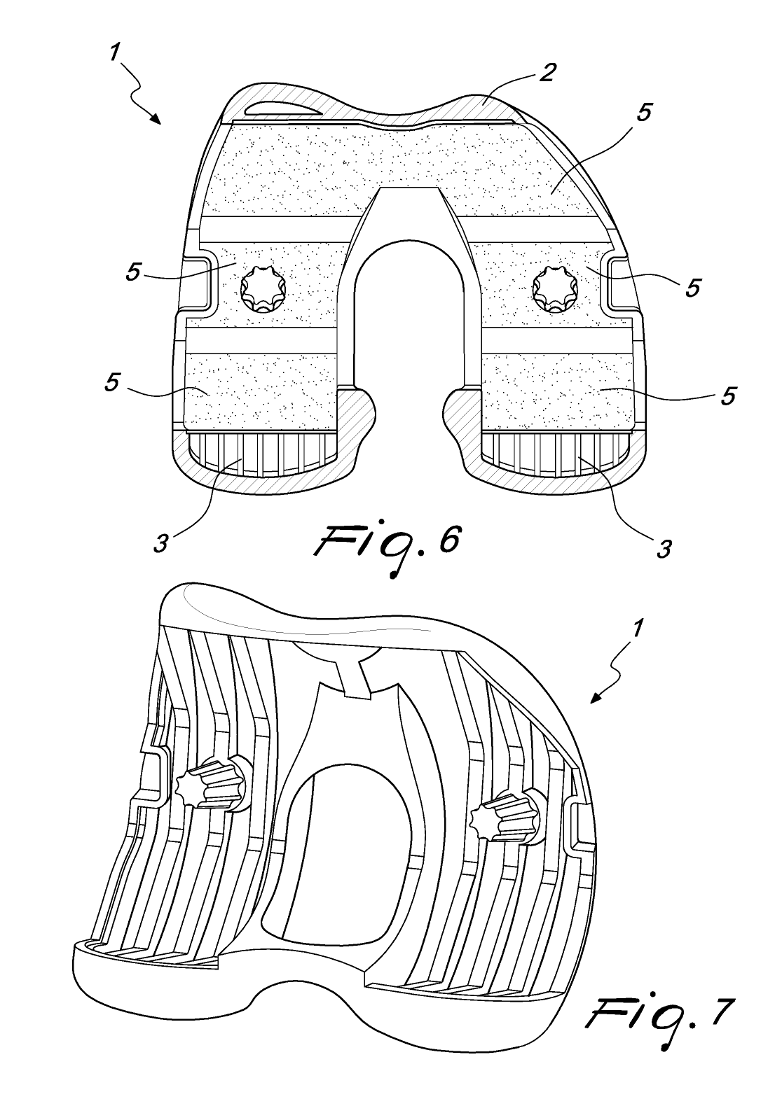

In a second embodiment, in which the femoral component is adapted to be applied by means of cement, the porous structure 4 of the first embodiment is replaced with a bone interface surface 5 (see FIG. 6) which is smooth, with a porous structure having a porosity of less than 20 microns.

With the embodiments cited above, it is therefore possible to provide a femoral component of the knee prosthesis that has a reduced weight but sufficient and adequate mechanical strength.

The femoral component thus conceived is susceptible of numerous modifications and variations. All the details may furthermore be replaced with other technically equivalent elements.

In practice, the materials used, as well as the contingent shapes and dimensions, may be any according to requirements and to the state of the art.

The disclosures in Italian Patent Application No. MI2013A002154 from which this application claims priority are incorporated herein by reference.

1-6. (canceled)

7. A femoral component for cementless knee prostheses, comprising a body of the femoral component that defines a supporting structure of the femoral component with mediolateral or longitudinal reinforcements,
a highly porous structure arranged inside the body of the femoral component,
and a porous structure arranged as an interface between the femoral component and a bone,
said femoral component being provided by means of EBM beam or laser beam powder technology.

8. A femoral component for knee prostheses with cement, comprising a body of the femoral component that is adapted to define a supporting structure of the femoral component with mediolateral or longitudinal reinforcements,
a highly porous structure arranged inside said body,
and a substantially smooth structure arranged over the porous structure and adapted to define an interface with a bone,
said femoral component being provided by means of EBM beam or laser beam powder technology.

9. The femoral component according to claim 7, wherein said porous structure is arranged as an interface between the femoral component, and the bone has a porosity comprised between 300 and 1000 microns.

10. The femoral component according to claim 7, wherein the femoral component is adapted to be mated with said bone without cement.

11. The femoral component according to claim 8, wherein the femoral component is adapted to be mated with said bone by using cement.

12. The femoral component according to claim 7, wherein said highly porous structure has a porosity of more than 1000 microns.

13. The femoral component according to claim 8, wherein said highly porous structure has a porosity of more than 1000 microns.
